(19) United States
(12) Patent Application Publication
(10) Pub. No.: US 2023/0047027 A1
(43) Pub. Date: Feb. 16, 2023

(54) COMPOSITIONS AND METHODS USING CANNABINOID COMPOUNDS FOR REGULATING GASTRIC ACID SECRETION TO TREAT GASTROESOPHAGEAL REFLUX DISEASE AND RELATED CONDITIONS

(71) Applicant: Demetrix, Inc., Berkeley, CA (US)

(72) Inventors: Cynthia W. Byrant, Raleigh, NC (US); Alison Watta, Durham, NC (US)

(21) Appl. No.: 17/864,095

(22) Filed: Jul. 13, 2022

Related U.S. Application Data
(60) Provisional application No. 63/221,473, filed on Jul. 13, 2021.

(51) Int. Cl. A61K 31/05

(52) U.S. Cl. CPC A61K 31/05

(57) ABSTRACT

Compositions for regulating gastric acid buildup using cannabinoid compounds are described herein. The cannabinoid compounds can include one or more of cannabicyclol (“CBL”), cannabichromene (“CBC”), cannabidivarin (“CBDV”), cannabidol (“CBD”), cannabigerol hexyl (“CBG-C6”), cannabigerol heptyl (“CBG-C7”), cannabigerol nonyl (“CBG-C9”), cannabidol-C2 (“CBD-C2”), cannabidol hexyl (“CBD-C6”), and cannabidol heptyl (“CBD-C7”). The cannabinoid compounds can be H₂ antagonists. Methods and articles including the cannabinoid compounds are further disclosed.

### CROSS-REFERENCE TO RELATED APPLICATIONS

The present disclosure claims the priority benefit of U.S. Provisional Patent App. Ser. No. 63/221,473, filed Jul. 13, 2021, which is hereby incorporated herein by reference.

### TECHNICAL FIELD

The present disclosure generally relates to the use of cannabinoid compounds and related products to alleviate symptoms and treat conditions caused by the buildup of gastric acid, including gastroesophageal reflux disease (“GERD”). The cannabinoid compounds can be histamine H₂-receptor antagonists (“H₂ antagonists”).

### BACKGROUND

Blockage and buildup of gastric acid in the stomach of individuals can cause a variety of painful conditions including gastroesophageal reflux disease (“GERD”), gastric and duodenal ulcers, gastric hypersecretion, indigestion, and heartburn. Gastric acid buildup can be treated with H₂ antagonists which suppress the secretion of gastric acid from parietal cells located in the stomach. Suppression of gastric acid secretion can alleviate symptoms caused by gastric acid buildup including GERD.

### SUMMARY

According to one embodiment, a composition includes one or more cannabinoid compounds selected from the group including cannabicyclol (“CBL”), cannabichromene (“CBC”), cannabidivarin (“CBDV”), cannabidol (“CBD”), cannabigerol hexyl (“CBG-C6”), cannabigerol heptyl (“CBG-C7”), cannabigerol nonyl (“CBG-C9”), cannabidol-C2 (“CBD-C2”), cannabidol hexyl (“CBD-C6”), and cannabidol heptyl (“CBD-C7”). The composition regulates the secretion of gastric acid in the stomach.

According to another embodiment, a composition includes one or more cannabinoid compounds and regulates the secretion of gastric acid in the stomach. The one or more cannabinoid compounds are histamine H₂ receptor antagonists.

According to another embodiment, a method of regulating the secretion of gastric acid in the stomach includes administering a therapeutically effective amount of one or more cannabinoid compounds selected from the group including cannabicyclol (“CBL”), cannabichromene (“CBC”), cannabidivarin (“CBDV”), cannabidol (“CBD”), cannabigerol hexyl (“CBG-C6”), cannabigerol heptyl (“CBG-C7”), cannabigerol nonyl (“CBG-C9”), cannabidol-C2 (“CBD-C2”), cannabidol hexyl (“CBD-C6”), and cannabidol heptyl (“CBD-C7”).

According to another embodiment, a method of regulating the secretion of gastric acid in the stomach includes administering a therapeutically effective amount of one or more cannabinoid compounds. The one or more cannabinoid compounds are histamine H₂ receptor antagonists.

### DETAILED DESCRIPTION

Gastroesophageal reflux disease (“GERD”) is a chronic and painful condition caused by gastric acid buildup and causes symptoms including heartburn, chest pain, regurgitation, breathing problems, acidic taste, halitosis, and tooth damage. Compositions, articles, products, methods and treatments to reduce or eliminate gastric acid buildup using cannabinoid compounds are described herein. In certain embodiments, the cannabinoid compounds described herein can alleviate gastric acid secretion by acting as H₂ antagonists. H₂ antagonists (also called H₂ blockers) are compounds which exhibit antagonistic, or blocking, activity against the effects of histamine on the histamine H₂ G protein-coupled receptor (“GPCR”).

As can be appreciated, H₂ receptors are known to regulate gastric acid secretion by parietal cells. Specifically, H₂ receptors in the stomach trigger the secretion of gastric acid upon activation with histamine. Histamine H₂ receptor antagonists (“H₂ antagonists”) can reduce gastric acid secretion by parietal cells by dampening their activation caused by the histamine and thereby reduces both stimulated and basal gastric acid secretion. Known H₂ antagonists are effective in the treatment of GERD and other conditions caused by excessive gastric acid secretion and buildup including dyspepsia, peptic ulcers, and Zollinger-Ellison syndrome.

However, known H₂ antagonists such as cimetidine, ranitidine, famotidine, nizatidine, roxatidine, lafutidine, lavotidine, and niperotidine, which are synthesized drugs, suffer from a variety of issues. For example, these known synthetic drugs can have adverse side effects and some suffer from various contamination issues due to their synthetic production routes. Additionally, tolerance to H₂ antagonists can also develop leading to ineffective treatment with such synthetic drugs. However, such issues with H₂ antagonists can be reduced or eliminated by using the cannabinoid compounds and related products described herein.

Cannabinoid compounds discovered to act as H₂ antagonists include cannabicyclol (“CBL”), cannabichromene (“CBC”), cannabidivarin (“CBDV”), and cannabidol (“CBD”) as well as variants of cannabinoids having alkyl side chains of varying lengths including cannabigerol hexyl (“CBG-C6”), cannabigerol heptyl (“CBG-C7”), cannabigerol nonyl (“CBG-C9”), cannabidol-C2 (“CBD-C2”), cannabidol hexyl (“CBD-C6”), and cannabidol heptyl (“CBD-C7”).

However, it has also been found that certain cannabinoid compounds do not act as H₂ antagonists and are not expected to be effective in reducing the amount of gastric acid secreted into the stomach. Examples of such cannabinoid compounds include cannabigerol (“CBG”), cannabigerivarin (“CBGV”), cannabidivarinic acid (“CBDVA”), cannabinol (“CBN”), cannabichromenic acid (“CBCA”), cannabicyclolic acid (“CBLA”), tetrahydrocannabivarin (“THCV”), cannabidiolic acid (“CBDA”), and cannabigerolic acid (“CBGA”). Variants of cannabinoids that did not act as H₂ antagonists include (+)-cannabidiol (“(+)-CBD”), cannabidiorcol (“CBD-C1”), cannabidol nonyl (“CBD-C9”), cannabigerolic acid hexyl (“CBGA-C6”), cannabigerolic acid heptyl (“CBGA-C7”), cannabigerolic acid nonyl (“CBGA-C9”), cannabigerolic acid butyl (“CBGA-C4”), cannabigerovarinic acid (“CBGVA”) and cannabigerol butyl (“CBG-C4”).

Prior to the present discovery, it was not appreciated which specific cannabinoid compounds could act as H₂ antagonists. For example, prior attempts at addressing gastric acid issues such as GERD did not focus on specific cannabinoid compounds and did not have or rely on the H₂ receptor specificity as described in the present disclosure. By selecting only cannabinoid compounds that act as H₂ antagonists, the methods, compositions and products described herein can provide improved treatment of gastric acid conditions with targeted specificity.

It has additionally been discovered that the alkyl side chain length of cannabinoid compounds can influence the effect the cannabinoid compounds have on the GPCR receptors. For example, it has been discovered that increasing the length of the alkyl side chain, generally increases the antagonism of CBG and CBD to the H₂ receptor. Although trends are observed for the chain length of the cannabinoids, chain length can also be unpredictable as CBD was observed to generally increase in inhibition against the H2 receptor with increasing chain length, but had an outlier where CBD-C9 did not act as an antagonist. The trend is reinforced by the RC₅₀ data which confirms that cannabinoid compounds with longer alkyl chains reach the RC₅₀ at smaller concentrations even when such cannabinoids do not inhibit the H₂ receptor as strongly as tiotidine. The RC₅₀ value indicates the value at which the cannabinoid reached the reference compound's 50% effective concentration.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to treat gastric acid diseases with specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally-derived cannabinoid compounds offer benefits in regard to the treatment of gastric acid buildup not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of alleviating excess gastric acid that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally extracted cannabinoid compounds are sufficiently pure and/or if any containments are sufficiently well understood.

By acting as H₂ antagonists, the cannabinoid compounds described herein can reduce or eliminate the symptoms caused by excessive gastric acid buildup and can treat GERD and other related diseases and conditions. The symptoms caused by gastric acid buildup can be reduced in intensity, duration, type, and/or inducement. In certain embodiments, the compositions or products containing the herein described cannabinoid compounds can be taken on a predetermined routine schedule to treat chronic gastric acid buildup. Additionally, or alternatively, such compositions or products can be taken on an as-needed schedule to regulate the secretion of gastric acid and treat any gastric acid buildup.

Generally, reduction in the amount of gastric acid in the stomach can be accomplished by treatment with a therapeutically effective amount of one or more of CBL, CBC, CBDV, CBD, CBG-C6, CBG-C7, CBG-C9, CBD-C2, CBD-C6, and CBD-C7. In certain embodiments, each of CBL, CBC, CBDV, CBD, CBG-C6, CBG-C7, CBG-C9, CBD-C2, CBD-C6, and CBD-C7 can be included in a composition or article to reduce the production of gastric acid while in other embodiments, various combinations and subsets of CBL, CBC, CBDV, CBD, CBG-C6, CBG-C7, CBG-C9, CBD-C2, CBD-C6, and CBD-C7 can be included in such compositions and articles.

For example, in certain embodiments, only one cannabinoid compound, such as CBC, can be included. As can be appreciated however, any combination CBL, CBC, CBDV, CBD, CBG-C6, CBG-C7, CBG-C9, CBD-C2, CBD-C6, and CBD-C7 can be effective including the use of just a single cannabinoid compound selected from the foregoing cannabinoid compounds.

A therapeutically effective amount of the one or more cannabinoid compounds can vary depending on factors such as the desired effect of treatment, the severity of the gastric acid buildup (e.g., the severity of GERD or other symptoms), the duration of treatment, or the method of delivering the cannabinoid compounds to the subject. For example, to alleviate GERD caused by consumption of a specific food may require a different amount, or dosage, of the cannabinoid compounds than the amount required to treat GERD caused by chronic overproduction of gastric acid.

In certain embodiments, a therapeutically effective amount for internal use (e.g., an oral or injectable composition) can be about 100 mg of the cannabinoid compounds or less; in certain embodiments, about 75 mg of the cannabinoid compounds or less; in certain embodiments, about 50 mg of the cannabinoid compounds or less; in certain embodiments, about 20 mg of the cannabinoid compounds or less; in certain embodiments, about 10 mg of the cannabinoid compounds or less; in certain embodiments, about 5 mg of the cannabinoid compounds or less; in certain embodiments, about 1 mg of the cannabinoid compounds or less; in certain embodiments, about 500 μg of the cannabinoid compounds or less; in certain embodiments, about 100 μg of the cannabinoid compounds or less; and in certain embodiments, about 500 pg of the cannabinoid compounds or less.

As can be appreciated, the relative concentration of the cannabinoid compounds can vary in different compositions and products. For example, a beverage containing the cannabinoid compounds can have a smaller concentration of the cannabinoid compounds than a pill or capsule. In certain embodiments however, the total amount of the cannabinoid compounds can be the same between such two compositions and articles. In other embodiments, both the concentration and amount of cannabinoid compounds can vary between different compositions and articles.

In certain embodiments, the relative amounts of each of CBL, CBC, CBDV, CBD, CBG-C6, CBG-C7, CBG-C9, CBD-C2, CBD-C6, and CBD-C7 can vary in the compositions and articles described herein. For example, each individual cannabinoid compound (CBL, CBC, CBDV, CBD, CBG-C6, CBG-C7, CBG-C9, CBD-C2, CBD-C6, and CBD-C7) can vary from each other cannabinoid compound by about 1,000:1 to about 1:1,000. As can be appreciated, the amount and ratios of each of the cannabinoid compounds can be selected based on factors such as the method of delivery, the amount of gastric acid buildup being treated, and individual factors such as the body weight of person consuming the cannabinoid compounds.

In certain embodiments, the compositions, articles, and methods described herein can be substantially or entirely free of cannabinoid compounds other than CBL, CBC, CBDV, CBD, CBG-C6, CBG-C7, CBG-C9, CBD-C2, CBD-C6, and CBD-C7. For example, the compositions, articles, and methods can be substantially or entirely free of CBG, CBGV, CBDVA, CBN, CBCA, CBLA, THCV, CBDA, CBGA, CBGVA, CBGA-C4, CBG-C4, CBD-C1, (+)-CBD, CBD-C9, CBGA-C6, CBGA-C7, CBGA-C9, and tetrahydrocannabinol (“THC”). As used herein, substantially free can mean less than about 5%, less than about 4%, less than about 3%, less than about 2%, less than about 1%, less than about 0.5%, less than about 0.1%, or less than about 0.01%. In certain embodiments, the cannabinoid compounds can be produced using non-horticulturally-derived methods such as through chemical synthesis (e.g., organic synthesis reactions) or through modification of yeast and/or bacterial cells to produce the cannabinoid compounds in high purity. However, in certain embodiments, cannabinoid compounds can also be a natural product, e.g., an extract of a cannabis plant if sufficiently pure. In certain embodiments, substantially pure means that the isolated cannabinoid compound, when added, includes about 3% or less of contaminants, about 2% or less of contaminants, about 1% or less of contaminants, about 0.5% or less of contaminants, about 0.1% or less of contaminants, or about 0.01% or less of contaminants.

In certain embodiments, the compositions, articles, and methods described herein can be utilized on a predetermined schedule (e.g., nightly, twice daily, etc.) or can be utilized on an as-needed basis. In certain embodiments, the predetermined schedule can be based on the half-life of the cannabinoid compounds as well as the release dynamics of the cannabinoid compounds. As can be appreciated, it can be useful in certain embodiments, to release the cannabinoid compounds described herein using a delayed release mechanism, such as a delayed release pill, to regulate the bioavailable amounts of the cannabinoid compounds.

In certain embodiments, the cannabinoid compounds described herein can be included in a composition or article to partially or fully alleviate diseases or symptoms caused by gastric acid buildup. The composition or article can be consumed by, or be applied onto, a person to alleviate the buildup of gastric acid. Generally, the exact nature of the composition or article can vary widely.

For example, the cannabinoid compounds can be included in pills or capsules that can be taken quickly and efficiently on a regular or as needed basis (daily, with meals, etc.). Use of an oral pill or capsule can be useful in the treatment of GERD. As can be appreciated, pills and capsules can contain a number on inactive ingredients as known in the art such as dicalcium phosphate dehydrate, microcrystalline cellulose, stearic acid, silicon dioxide, croscarmellose sodium, magnesium stearate, and pharmaceutical glaze. Other known pills and capsules are also contemplated herein. As an additional example, a compressed chewable tablet can include a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), the cannabinoid compounds and, optionally, a lubricant (such as magnesium stearate, stearic acid, talc, and waxes), sweetening, coloring and flavoring agents, a surfactant, a preservative, and other ingredients. All of the ingredients, including the one or more cannabinoid compounds, are dry blended and compressed into a tablet.

In certain embodiments, the cannabinoid compounds can alternatively be administered to individuals via food products and other comestibles. By way of illustration and not as a limitation, the selected cannabinoid compounds can be incorporated into a beverage, a “smoothie” (fruit, vegetable, nut oil, or yogurt based), a frozen desert (e.g., ice cream or sorbet), a food bar, a nutrition bar, a dressing, a snack, into a flour- or flour-alternative-based product, a rice-based product, pastes, gels, powders, gums, etc. Incorporation into food products can facilitate consumption of the cannabinoid compounds and increase palatability.

As can be appreciated, the exact nature of the food article can influence the bioavailability of the cannabinoid compounds. For example, a cannabinoid included in a large food article may take more time to become bioavailable than the same amount of cannabinoid compounds in a single pill or capsule. Generally, the remainder of the composition or article can constitute any suitable non-bioactive component such as filler, food, or water.

In certain embodiments, the compositions or articles including the cannabinoid compounds described herein can include indicia and/or packaging to convey to end users the amount of the cannabinoid compounds contained therein. For example, a small nutrient bar may be individually labeled and packaged to express to the end user that only a single bar should be consumed.

As will be appreciated, a wide variety of different compositions and articles can be prepared which include the one or more cannabinoid compounds of the present disclosure including compositions and articles not listed here. All such compositions and articles are contemplated herein as they are within the ordinary skill of artisans based on the guidance provided in the present disclosure.

Generally, all of the compositions and articles described herein can be manufactured and produced as known in the art. For example, in certain embodiments, the cannabinoid compounds can be dissolved in a suitable solvent such as an alcohol or oil and then added to the composition or article.

### EXAMPLES

To evaluate the H₂ antagonist activity of the cannabinoid compounds, a GPCR reactivity assay was performed. The evaluated cannabinoid compounds were: CBN, THCV, CBDVA, CBG, CBL, CBC, CBDV, CBDA, CBD, CBGA, (+)-CBD, CBG-C4, CBD-C1, CBD-C2, CBGV, CBGA-C4, CBGVA, CBCA, and CBLA. Tiotidine was used as the control.

To perform the GPCR reactivity assay, a commercial GPCR assay, PathHunter® (β-Arrestin from Eurofins DiscoverX Products (Fremont, Calif.), was used. In the PathHunter® (β-Arrestin GPCR assay, an inactive peptide fragment is fused to the targeted GPCR receptor and a complementary peptide fragment is fused to β-arrestin. At activation of the GPCR receptor and recruitment of β-arrestin, complementation of the peptide fragments occurs and restores β-galactosidase activity. The amount of β-galactosidase activity is then measurable using chemiluminescent reagents.

A single assay was run. In each run, PathHunter® cell lines were removed from a freezer stock and seeded at a volume of 20 μL into white walled, 384-well microplates and incubated at 37° C. Each cell was pre-incubated with the cannabinoid compound followed by an agonist challenge at the EC80 concentration. Cells were then diluted to generate a 5x sample in assay buffer. 5 μL of the 5x sample was then added to cells and incubated at 37° C. for 30 minutes. Finally, 5 μL of 6x EC80 agonist in assay buffer were added to the cells and incubated at 37° C. for 90 minutes or 180 minutes. The assay signal was generated through addition of 12.5 μL or 15 μL (50% V/V) of a detection reagent cocktail followed by a one hour incubation time at room temperature. Microplates were read following signal generation with a PerkinElmer Envision™ (PerkinElmer Inc., Waltham, Mass.) for chemiluminescent signal detection. Reactivity was analyzed using the CBIS data analysis suite (ChemInnovation, CA) where RLU refers to the raw measured values. The percent inhibition was calculated using the formula: % Inhibition=100%*(1−mean RLU of test sample−mean RLU of control)/(mean RLU of EC80 control−mean RLU of vehicle control).

The results of the GCPR reactivity screen for the H₂ receptor are depicted in Table 1. The percent inhibition is depicted. As used herein, strong activity against a receptor is considered a percent activity of about 60% or higher. Higher results for antagonism against the H₂ receptor are considered better.

TABLE 1

| H₂ | (+)- | | | CBD- | CBD- | | CBG- | | | |
|---|---|---|---|---|---|---|---|---|---|---|
| Receptor | CBD | CBD | CBDV | C2 | C1 | CBG | C4 | CBGV | CBC | CBL |
| Antagonist | 45% | 77% | 78% | 73% | 47% | 80% | −62% | 10% | 117% | 82% |
| Activity | | | | | | | | | | |

| H₂ | | | | | | CBGA- | | | |
|---|---|---|---|---|---|---|---|---|---|
| Receptor | CBN | THCV | CBDA | CBDVA | CBGA | C4 | CBGVA | CBCA | CBLA |
| Antagonist | 76% | −22% | 4% | 12% | −3% | 16% | 2% | 12% | 11% |
| Activity | | | | | | | | | |

As depicted in Table 1, CBN, CBG, CBL, CBC, CBDV, CBD, and CBD-C2 each demonstrated strong antagonist activity with an activation of 60% or greater. CBC demonstrating very strong antagonist activity at an activation of 117%. Conversely, THCV, CBDVA, CBDA, CBGA, (+)-CBD, CBD-C1, CBG-C4, CBGV, CBGVA, CBGA-C4, CBCA, and CBLA did not demonstrate H2 antagonist activity with certain cannabinoids (THCV, CBGA, and CBG-C4) actually increasing sensitivity to histamine.

Based on the results of the GPCR reactivity screen, and IC₅₀ antagonist activity screen was run against the H₂ receptor. The IC₅₀ antagonist activity screen was run for each of CBG, CBC, CBL, CBN, CBDV, CBD, CBD-C1, CBD-C2, (+)-CBD, CBG-C6, CBG-C7, CBG-C9, CBD-C6, CBD-C7, CBD-C9, CBGA-C6, CBGA-C7, and CBGA-C9. IC₅₀ curves were measured using the same GPCR assay, PathHunter® β-Arrestin from Eurofins DiscoverX Products (Fremont, Calif.), as the GPCR reactivity screen. The maximum inhibition (antagonism) for each of the cannabinoids is reported in Table 2. As used herein, strong activity against a receptor is considered a maximum activity of about 60% or higher. Higher results for antagonism against the H₂ receptor are considered better.

TABLE 2

| | | IC50 (Inhibition - |
|---|---|---|
| Cannabinoid | RC50 (μM) | Max Response) |
| CBG | >10 | 21 |
| CBC | 2.63 | 62 |
| CBL | 1.17 | 64 |
| CBN | >10 | 12 |
| CBDV | 4.50 | 75 |
| CBD | 0.83 | 64 |
| CBD-C1 | >10 | 53 |
| CBD-C2 | 5.22 | 71 |
| (+)-CBD | >10 | 42 |
| CBG-C6 | >10 | 84 |
| CBG-C7 | 3.39 | 93 |
| CBG-C9 | 2.89 | 96 |
| CBD-C6 | 0.46 | 80 |
| CBD-C7 | 0.38 | 74 |
| CBD-C9 | 0.10 | 56 |
| CBGA-C6 | >10 | 9 |
| CBGA-C7 | >10 | 17 |
| CBGA-C9 | >10 | 11 |

The results depicted in Table 2 confirm the GPCR screening assay and confirm that cannabinoids including CBC, CBL, CBDV, CBD, and CBD-C2 exhibit an antagonistic effect on the H₂ receptor. Although CBN and CBG demonstrated antagonism to H₂ receptor in the GPRC screening assay, inhibition was not observed in the IC₅₀ data. Additionally, the IC₅₀ data shows that additional variants of such cannabinoids can also exhibit an antagonistic effect including CBG-C6, CBG-C7, CBG-C9, CBD-C6, and CBD-C7. In particular, the increasing alkyl side chain length is generally observed to increase the degree of antagonism to the H₂ receptor.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

1. A composition comprising one or more cannabinoid compounds selected from the group comprising cannabicyclol (“CBL”), cannabichromene (“CBC”), cannabidivarin (“CBDV”), cannabidol (“CBD”), cannabigerol hexyl (“CBG-C6”), cannabigerol heptyl (“CBG-C7”), cannabigerol nonyl (“CBG-C9”), cannabidol-C2 (“CBD-C2”), cannabidol hexyl (“CBD-C6”), and cannabidol heptyl (“CBD-C7”), and wherein the composition regulates the secretion of gastric acid in the stomach.

2. The composition of claim 1, wherein the one or more cannabinoid compounds are histamine H₂ receptor antagonists.

3. The composition of claim 1 comprises about 100 mg or less of the one or more cannabinoid compounds.

4. The composition of claim 1 is a liquid.

5. The composition of claim 1 is a food or beverage.

6. The composition of claim 1 is a pill or capsule.

7. The composition of claim 6 is a delayed-release pill or capsule.

8. The composition of claim 1, wherein the one or more cannabinoid compounds are non-horticulturally derived cannabinoid compounds.

9. The composition of claim 1 alleviates symptoms of one or more of gastroesophageal reflux disease (“GERD”), gastric and duodenal ulcers, gastric hypersecretion, indigestion, and heartburn.

10. The composition of claim 1 treats or prevents one or more of gastroesophageal reflux disease (“GERD”), gastric and duodenal ulcers, gastric hypersecretion, indigestion, and heartburn

11. A method of regulating the secretion of gastric acid in the stomach, the method comprising administering a therapeutically effective amount of one or more cannabinoid compounds selected from the group comprising cannabicyclol (“CBL”), cannabichromene (“CBC”), cannabidivarin (“CBDV”), cannabidol (“CBD”), cannabigerol hexyl (“CBG-C6”), cannabigerol heptyl (“CBG-C7”), cannabigerol nonyl (“CBG-C9”), cannabidol-C2 (“CBD-C2”), cannabidol hexyl (“CBD-C6”), and cannabidol heptyl (“CBD-C7”).

12. The method of claim 11, wherein the one or more cannabinoid compounds are histamine H₂ receptor antagonists.

13. The method of claim 11 alleviates the symptoms of gastroesophageal reflux disease (“GERD”).

14. The method of claim 11 alleviates the symptoms of one or more of gastric and duodenal ulcers, gastric hypersecretion, indigestion, and heartburn.

15. The method of claim 11 treats or prevents one or more of gastroesophageal reflux disease (“GERD”), gastric and duodenal ulcers, gastric hypersecretion, indigestion, and heartburn

16. The method of claim 11, wherein the one or more cannabinoid compounds are administered on a predetermined routine schedule.

17. The method of claim 11, wherein the one or more cannabinoid compounds are administered on an as-needed schedule.

18. The method of claim 11, wherein the one or more cannabinoid compounds are non-horticulturally derived cannabinoid compounds.
